(19) United States
(12) Patent Application Publication
(10) Pub. No.: US 2021/0361221 A1
(43) Pub. Date: Nov. 25, 2021

(54) METHODS AND DEVICES FOR MODULATING DELTA WAVES

(71) Applicant: Funnel Industries, LLC, Yorktown, VA (US)

(72) Inventor: Wesley Earl Miksa, Yorktown, VA (US)

(21) Appl. No.: 17/384,392

(22) Filed: Jul. 23, 2021

Related U.S. Application Data
(63) Continuation of application No. PCT/US20/15042, filed on Jan. 24, 2020.
(60) Provisional application No. 62/796,422, filed on Jan. 24, 2019.

(51) Int. Cl. A61B 5/374; A61B 5/383; A61B 5/291; A61B 5/00

(52) U.S. Cl. CPC A61B 5/374; A61B 5/4812; A61B 5/291; A61B 5/383

(57) ABSTRACT

Disclosed are methods and devices for modulating delta brainwaves in a sleeping subject. In various embodiments, the method includes a) detecting and monitoring an initial delta wave in a subject, b) applying an electrical current to modulate the initial delta wave in the subject, thereby modifying the initial delta wave in the subject to form a modified delta wave, and, optionally, repeating cycles of steps a) and b), thereby modulating delta brainwaves.

### CROSS-REFERENCE TO RELATED APPLICATIONS

This application is a continuation of International Application No. PCT/US2020/015042, filed Jan. 24, 2020, which claims the benefit of U.S. Provisional Patent Application No. 62/796,422, filed Jan. 24, 2019, each of which is hereby incorporated by reference in its entirety.

### BACKGROUND

Sleep restores biochemical processes and structures damaged or destroyed by radiation. Over 700 genes associated with physiological maintenance and restoration are activated during sleep. Effects of sleep deprivation include increased inflammation, impaired healing, increased risk of developing cancer, hormonal imbalances, loss of motor control and reaction time, memory and other cognitive impairments, emotional instability and anxiety, neurological and psychological disorders, reduced pain tolerance, increased risk of heart attack, diabetes, lessened respiratory and physical-mechanical function, immune system impairment, reduced effectiveness of vaccinations, visible aging, and eventual mortality. These effects are consistent with those of radiation exposure. Given the frequency and magnitude with which Earth's electromagnetic shield has likely declined with the planet's magnetic pole shifts in the past, ancestors of today's organisms were able to survive what was likely substantially greater radiation than is currently experienced on Earth today. Health benefits of sleep have appeared in literature for ages and sleep stages have been identified at the core of the generation and regeneration cycles in story structures of ancient and modern mythologies around the world. More recently, sleep has been prioritized to treat childhood social trauma now identified as a primary risk factor in reducing human longevity. Further, sleep pumps toxins from the brain.

Electrical current has been demonstrated to be central to healing processes, guiding bone healing and tissue regeneration and mediating the differentiation and dedifferentiation of cells. Electrical current controls velocity of blood cells, guides their direction of movement, and accelerates their dotting. Electrical current has been successfully applied to re-establish or activate nerve pathways in patients with severed spines, supporting restoration of walking ability. Electrical current, often of highly specific amperage, is a controlling factor in the regeneration of limbs in reptiles and amphibians.

The properties of the primitive non-rapid eye movement (NREM) deep sleep delta wave (delta wave) make it unique in its ability to activate genes affecting human whole-system physiological health. In various embodiments, the term ‘delta wave’ may be used interchangeably with ‘slow wave’ of slow wave sleep (SLS) or slow wave activity (SLA). The delta wave, with both the lowest frequency and highest intensity of all neurological electrical sleep activity, is uniquely capable of traveling the longest distances across the nervous system of all brainwaves and inducing restorative effects of sleep.

The sequential precedence of the emergence of NREM deep sleep over other sleep stages after periods of sleep deprivation suggests NREM deep sleep is the most critical sleep stage for physiological survival. The delta wave is the defining electrical feature of NREM deep sleep. Targeted suppression of the NREM deep sleep delta wave impacts whole system metabolic activity and disrupts cognitive function. Effective enhancement of the delta wave via entrainment with transcranial electrical stimulation was found to improve cognitive function and declarative memory. Targeted enhancement of the NREM deep sleep delta wave via entrainment with topically applied electrical current has been found to improve cognitive function and declarative memory in human test subjects. Release of human growth hormone, which affects the rate of healing processes, corresponds to delta wave activity. Onset of the delta wave's decline begins in mid-life when general physiological condition deteriorates and natural tooth loss accelerates in the absence of dentistry. Natural decline of the delta wave is correlated with decline in declarative memory consolidation and general physiological deterioration associated with aging. Total loss of the delta wave results in eventual death.

Some attempts to enhance the delta wave have failed. Application of a square wave (digital/binary) enhancement approach does not replicate cognitive benefit results achieved in research designs in which the delta wave was enhanced via analog input reinforcing its natural sinusoidal wave structure. Enhancing the delta wave for a temporary duration at the expense of total nighttime duration of the delta wave may entirely negate any positive effect. Clearly, novel devices and methods are needed to modulate the delta wave.

### BRIEF SUMMARY

The present disclosure is generally directed to methods and devices for modulating delta brainwaves (delta waves) in a sleeping subject.

In one aspect, a method for modulating delta brainwaves, comprising a) detecting and monitoring an initial delta wave in a subject; b) applying an electrical current to modulate the initial delta wave in the subject, thereby modifying the initial delta wave in the subject to form a modified delta wave; c) optionally repeating cycles of steps a) and b), thereby modulating delta brainwaves, is provided herein. In another aspect, a method for modulating delta brainwaves in a sleeping subject, comprising a) detecting and monitoring an initial delta wave in the sleeping subject; b) applying an electrical current to modulate the initial delta wave in the sleeping subject, thereby modifying the initial delta wave in the sleeping subject to form a modified delta wave; optionally repeating cycles of steps a) and b), thereby modulating delta brainwaves in the sleeping subject, is provided herein. In another aspect, a method for initiating and modulating delta brainwaves in a sleeping subject, comprising a) detecting and monitoring sleep metrics selected from the group consisting of heart rate, breathing rate, and body motion identifying onset of non-rapid eye movement (NREM) deep sleep and an initial delta wave in the sleeping subject; b) applying an electrical current to initiate formation of and to modulate the initial delta wave in the sleeping subject, thereby initiating and modifying the initial delta wave in the sleeping subject to form a modified delta wave; and c) optionally repeating cycles of steps a) and b), thereby initiating and modulating delta brainwaves in the sleeping subject, is provided herein.

In another aspect, a method of treating a disease or a condition, comprising modulating delta brainwaves in a subject in need thereof, comprising a) detecting and monitoring an initial delta wave in a subject; b) applying an electrical current to modulate the initial delta wave in the subject, thereby modifying the initial delta wave in the subject to form a modified delta wave; c) optionally repeating cycles of steps a) and b), thereby treating the disease or the condition, is provided herein. In still another aspect, a method of treating a disease or a condition, comprising modulating delta brainwaves in a sleeping subject in need thereof, comprising a) detecting and monitoring an initial delta wave in the sleeping subject; b) applying an electrical current to modulate the initial delta wave in the sleeping subject, thereby modifying the initial delta wave in the sleeping subject to form a modified delta wave; c) optionally repeating cycles of steps a) and b), thereby treating the disease or the condition, is provided herein.

In yet another aspect, a method of reducing or preventing a disease or a condition, comprising modulating delta brainwaves in a subject in need thereof, comprising a) detecting and monitoring an initial delta wave in a subject; b) applying an electrical current to modulate the initial delta wave in the subject, thereby modifying the initial delta wave in the subject to form a modified delta wave; c) optionally repeating cycles of steps a) and b), thereby reducing or preventing the disease or the condition, is provided herein.

In another aspect, a device, comprising a) at least one sensor to monitor brainwave activity including a delta wave; b) an amplifier and control board; c) a negative electrode; d) a positive electrode; and e) an electrical stimulator to modulate delta brainwaves, is provided herein. In yet another aspect, a device, comprising a) at least one sensor to monitor sleep metrics selected from the group comprising heart rate, breathing rate, and body motion and sleep brainwave activity including a delta wave; b) an amplifier and control board; c) a negative electrode; d) a positive electrode e) an electrical stimulator to modulate sleep brainwaves, is provided herein.

### BRIEF DESCRIPTION OF THE FIGURES

FIG. 1 shows device illustration of Overcoming Solar and Interstellar Radiation Impacts with Sleep-Brainwave Entrainment (OSIRIS-BE) Device.

FIG. 2 shows device illustration of OSIRIS-BE Device in a compact version that does not involve external wires.

FIG. 3 depicts a computing node according to an embodiment of the present disclosure.

### DETAILED DESCRIPTION

The present disclosure is generally directed to methods and devices for modulating delta brainwaves in a sleeping subject.

In some embodiments, the amplitude, wave structure, and duration of sleep brainwaves of an individual have been found to be associated with the individual's cognitive and physiological health. In some embodiments, the device is a pocket-sized, standalone portable unit that operates on a battery with minimal operation and instruction required by the subject. In some embodiments, the function of the device is to restore to a healthy condition the delta wave in a subject. In some embodiments, the device actively monitors the subject's brainwaves with sensors, processes the monitoring data, and applies information from the monitoring data to adjust in real-time targeted electrical currents from electrodes topically applied to the subject's head, toward the maintenance and restoration of the subject's brainwaves during sleep to a healthy condition. In some embodiments, electrical inputs applied topically from the device utilize physical principles of wave entrainment, phase-amplitude coupling (PAC), and electrical stimulation (similar to those of pacemakers for the heart, automated external defibrillators (AEDs), or electrical muscle stimulators) to support the restoration and maintenance of healthy amplitude, wave structure, and duration of sleep brainwaves, such as the low frequency non-rapid eye movement (NREM) deep sleep delta wave, in the user. In some embodiments, when the delta wave in the subject is not detectable the device actively monitors other sleep metrics of heart rate, breathing rate, and body motion and applies electrical current to return to existence and actively monitor the delta wave and applies electrical current to restore to healthy condition the delta wave in the subject. In some embodiments, the unmodified delta wave in the subject should be present in the subject coinciding with heart rate, breathing rate, or body motion but is non-detectable with an electroencephalogram (EEG) in the subject. In some embodiments, the initial delta wave is non-detectable with an EEG in the subject. In some embodiments, the unmodified delta wave in the subject should be present in the subject coinciding with heart rate, breathing rate, or body motion but is non-detectable with an EEG in the subject and only becomes detectable in the subject with an EEG upon initiation by stimulation with electrical current. In some embodiments, the modified delta wave is detectable in the subject with an EEG.

In some embodiments, the OSIRIS-BE Device (described in further detail below) is worn and turned on before and throughout sleep. The OSIRIS-BE Device is turned off and removed upon awakening. In some embodiments, the primary function of the OSIRIS-BE Device is to monitor the user's NREM deep sleep delta wave using the sensors and to use the monitoring data to apply the appropriate amounts of electrical current at the proper times to restore the user's NREM deep sleep delta wave to healthy condition in terms of the wave's intensity, wave structure, and duration. In some embodiments, for users who have no detectable NREM deep sleep delta wave, the device operates to provide electrical inputs that stimulate return to measurable detection of and modulation of the NREM deep sleep delta wave to become more similar to the delta wave of a healthy individual.

In some embodiments, the benefits of the enhancement of delta brainwaves are cumulative and increase over days, weeks, and months of use. In some embodiments, because the delta wave tends to follow the path of other brainwaves formed during wakefulness and consolidates the gains of waking activity associated with the other brainwaves, diversifying physical, cognitive, and social activities during wakefulness is likely to further improve physiological and cognitive results by increasing neurons available for delta wave entrainment and support of the delta wave.

In some embodiments, the full benefits of the OSIRIS-BE Device can be gradual and occur after several weeks or months of use, as in the case of individuals impacted by the accumulation of excessive neurotoxins in the brain, nervous system damage, exposure to adverse environmental factors, participation in activities that can increase the likelihood of physiological or psychological damage or disease or other factors that can adversely impact brainwave activity and the restorative effects brainwave activity induces, including medical or psychological conditions that can overwhelm the central nervous system (CNS) or otherwise interfere with brainwave activity. In some embodiments, the toxins may include those responsible for the development of the proteins and amyloid plaques responsible for Alzheimer's and Parkinson's diseases. In some embodiments, the glymphatic system is supported in removing toxins from the brain via enhanced pumping and flushing actions of cerebrospinal fluid and interstitial fluid, and accelerated unidirectional flows or pulsatile motions, resulting from electrical input to improve constriction and expansion of blood vessels and extracellular spaces referred to here as neuroglymphatic electrical stimulation (NGES). In some embodiments, the electrical input and resulting stimulation provided by the OSIRIS-BE device aids in this pumping and flushing action, which can require weeks or months of use of the OSIRIS-BE Device to realize the full effects of the enhanced toxin removal. In some embodiments, the removal of the toxins further aids in re-establishing the health of the NREM deep sleep delta wave, as the toxins themselves interfere with the wave. In some embodiments, weakened neurons from tissue damage or disease are stimulated by use of the OSIRIS-BE Device. In some embodiments, new neurons otherwise uninvolved with delta wave activity are stimulated and recruited over time by use of the OSIRIS-BE Device to support the user's delta wave. Therefore, with some users the full benefit of use of the OSIRIS-BE Device is realized after weeks or months of use as toxins are removed from the user's brain. with some users the full benefit of use of the OSIRIS-BE Device is realized after weeks or months of use as weakened neurons or new neurons are stimulated and recruited by regular use of the OSIRIS-BE Device to support the user's delta wave.

In some embodiments, the technology specifically targets the delta wave for reconstructing the wave's intensity, structure, and duration during NREM deep sleep when the delta wave is known to naturally emerge in young, healthy individuals. In some embodiments, the technology monitors the person's physiological state, the real-time condition of their delta wave at the sleep stages in which the delta wave would appear in a young health individual, and provides electrical current of the proper type and at the proper time to reconstruct a healthy delta wave in the individual similar to that of a young healthy individual. In some embodiments, the system monitors brainwaves for the emergence of low frequency (˜0.1 Hz to 4 Hz, and as high as 20 Hz), high amplitude (generally above 75 microvolts [μV], but also sometimes as low as less than 10 μV and greater than 140 μV, as detected topically on the skin) brainwaves during sleep. In some embodiments, the system applies an algorithm to identify the delta wave based on the monitoring data, assess the delta wave's health based on the data (frequency in Hz, intensity in μV as measured topically on skin, and wave structure integrity), identify its healthy or ideal natural best-state based on the assessment, and input electrical current to gradually entrain the delta wave back to its healthy or ideal natural best-state during the user's non-REM deep sleep cycles. In some embodiments, the duration of the monitored delta wave activity and the duration of electrical input is timed by the technology during each sleep session such that the total combined duration of electrical input for each sleep session never exceeds 2 hours and the electrical input for each non-REM deep sleep cycle never exceeds a total duration of 40 minutes. In some embodiments, in order to not interfere with the progression of sleep cycles through natural sleep stages (non-REM through REM and back to non-REM) the intensity of electrical input also diminishes and eventually ceases as the measured delta wave diminishes and becomes undetectable toward the end of the sleep stage. The electrical input once again begins to be applied as the delta wave becomes detectable when the user cycles into the next non-REM deep sleep stage. In some embodiments, in order to avoid interference between inputs to the technology (monitoring data) and outputs from the technology (electrical current), the technology does not output electrical current at the same time it is receiving input and instead alternates between providing output and receiving input. In some embodiments, the algorithm adjusts the output from the technology between each output/input cycle based on effectiveness of the technology's output in achieving intended results based on input. In some embodiments, the outputted electrical current of the system matches the frequency of the user's delta wave and is designed to rise and fall (in analog/sine wave, not digital square wave) in amplitude at this frequency slightly above the amplitude of the user's measured delta wave, adjusting for current loss between brain and skin surface, in order to gradually entrain the delta wave to healthy or ideal natural best-state in sync with progression through sleep cycles during each sleep session (full sleep period). In some embodiments, the technology is informed by research pointing to cognitive and whole-system physiological health benefits of a healthy delta wave. In some embodiments, system physiological benefits of delta wave enhancement are directly or indirectly neurological, cognitive, psychological, metabolic, immunological, and restorative to tissues.

In certain aspects, disclosed herein is a method for modulating delta brainwaves, comprising a) detecting and monitoring an initial delta wave in a subject; b) applying an electrical current to modulate the initial delta wave in the subject, thereby modifying the initial delta wave in the subject to form a modified delta wave; c) optionally repeating cycles of steps a) and b), thereby modulating delta brainwaves. In another aspect, a method for modulating delta brainwaves in a sleeping subject, comprising a) detecting and monitoring an initial delta wave in the sleeping subject; b) applying an electrical current to modulate the initial delta wave in the sleeping subject, thereby modifying the initial delta wave in the sleeping subject to form a modified delta wave; optionally repeating cycles of steps a) and b), thereby modulating delta brainwaves in the sleeping subject, is provided herein. In another aspect, a method for initiating and modulating delta brainwaves in a sleeping subject, comprising a) detecting and monitoring sleep metrics selected from the group consisting of heart rate, breathing rate, and body motion identifying onset of non-rapid eye movement (NREM) deep sleep and an initial delta wave in the sleeping subject; b) applying an electrical current to initiate formation of and to modulate the initial delta wave in the sleeping subject, thereby initiating and modifying the initial delta wave in the sleeping subject to form a modified delta wave; and c) optionally repeating cycles of steps a) and b), thereby initiating and modulating delta brainwaves in the sleeping subject, is provided herein.

In another aspect, a method of treating a disease or a condition, comprising modulating delta brainwaves in a subject in need thereof, comprising a) detecting and monitoring an initial delta wave in a subject; b) applying an electrical current to modulate the initial delta wave in the subject, thereby modifying the initial delta wave in the subject to form a modified delta wave; c) optionally repeating cycles of steps a) and b), thereby treating the disease or the condition, is provided herein. In still another aspect, a method of treating a disease or a condition, comprising modulating delta brainwaves in a sleeping subject in need thereof, comprising a) detecting and monitoring an initial delta wave in the sleeping subject; b) applying an electrical current to modulate the initial delta wave in the sleeping subject, thereby modifying the initial delta wave in the sleeping subject to form a modified delta wave; c) optionally repeating cycles of steps a) and b), thereby treating the disease or the condition, is provided herein.

In yet another aspect, a method of reducing or preventing a disease or a condition, comprising modulating delta brainwaves in a subject in need thereof, comprising a) detecting and monitoring an initial delta wave in a subject; b) applying an electrical current to modulate the initial delta wave in the subject, thereby modifying the initial delta wave in the subject to form a modified delta wave; c) optionally repeating cycles of steps a) and b), thereby reducing or preventing the disease or the condition, is provided herein. In some embodiments, benefits of using the system described herein may include, but are not limited to, preventing, reducing, treating, and increasing resilience against diseases and adverse health conditions. In some embodiments, the system may monitor the delta wave and input electrical current in response to the results of the monitoring to restore the delta wave to healthy condition. In some embodiments, duration of electrical current input, amperage of electrical current input, and location of the electrical current input (topically above bridge of nose between frontal lobes) may be directed toward the same purpose: maximizing the health of the delta wave. In some embodiments, by maximizing the health of the delta wave, disease and adverse health conditions are both prevented and treated. In some embodiments, by maximizing the health of the delta wave, the user of the device becomes more resilient against physiological damage from adverse environmental conditions such as radiation exposure or more resilient against developing conditions such as CTE from chronic brain injuries.

In still another aspect, a method of treating a disease or a condition by restoring the delta wave of a subject in whom an initial delta wave is no longer detectable, comprising modulating delta brainwaves in a sleeping subject in need thereof, comprising a) detecting and monitoring sleep metrics selected from the group consisting of heart rate, breathing rate, and body motion and the initial delta wave; b) applying an electrical current to initiate formation of and to modulate the initial delta wave in the sleeping subject, thereby initiating and modifying the delta wave in the sleeping subject to form a modified delta wave; c) optionally repeating cycles of steps a) and b), thereby treating the disease or the condition, is provided herein. In still another aspect, a method treating a disease or a condition by restoring the delta wave comprising modulating delta brainwaves in a sleeping subject in need thereof, comprising a) detecting and monitoring sleep metrics selected from the group consisting of heart rate, breathing rate, and body motion and the initial delta wave; b) applying an electrical current to initiate formation of and to modulate the initial delta wave in the sleeping subject, thereby initiating and modifying the delta wave in the sleeping subject to form a modified delta wave; c) optionally repeating cycles of steps a) and b), thereby treating the disease or the condition. In step b), in some embodiments, the electrical current from the device is applied by the device once the sleep metrics data have adequately identified the non-REM deep sleep stages (Stages 3 and 4) as separate from the REM sleep stages, and the device has not been able to detect a delta wave sufficiently to identify its frequency in the user. In some embodiments, the electrical current output from the device to the user is provided at the end of Stage 3 and at Stage 4 sleep and cycles through frequencies (from low to high) and intensities (from low to high), with each output cycle beginning at ˜0.1 Hz from low to rising voltage, and the next output cycle beginning at ˜0.2 Hz from low to rising voltage, and so on up to ˜4 Hz. In some embodiments, the device monitors brainwave activity from the user after each output cycle from the device to assess responsiveness of the user's brainwave activity to the device's output in order to identify the best-fit frequency. In some embodiments, best-fit frequency is identified by measured duration and intensity of delta wave activity from the user following each device output cycle, with the longest duration and highest intensity brainwave feedback from the user corresponding to the user's proper delta wave frequency. In some embodiments, in the absence of a discernable best-fit frequency based on these cycles, the device initially provides output at a standard frequency of 0.5 Hz and continues to monitor for delta wave activity from the user, adjusting device output accordingly.

In another aspect, a device, comprising a) at least one sensor to monitor brainwave activity including a delta wave; b) an amplifier and control board; c) a negative electrode; d) a positive electrode; and e) an electrical stimulator to modulate delta brainwaves, is provided herein. In yet another aspect, a device, comprising a) at least one sensor to monitor sleep brainwave activity including a delta wave; b) an amplifier and control board; c) a negative electrode; d) a positive electrode e) an electrical stimulator to modulate sleep brainwaves, is provided herein.

In yet another aspect, a device, comprising a) at least one sensor (e.g., EEG sensor) to monitor brainwave activity including a delta wave; b) at least one sensor to monitor health metrics selected from the group consisting of heart rate, breathing rate, and body motion; c) an amplifier and control board; d) a negative electrode; e) a positive electrode; and f) an electrical stimulator to initiate and modulate delta brainwaves, is provided herein. In some embodiments, the sensor is a standard EEG sensor. In some embodiments, the sensor is placed on the forehead at or in close proximity to the location of the electrode (−) for electrical input: topically above the bridge of the nose at the location between the frontal lobes of the brain. In some embodiments, because the sensor is intended to monitor electrical activity from the brain, the sensor may be configured to not receive interference from the electrode (−) providing input. In some embodiments, the sensor is not receiving data at the moment the device is providing output. In some embodiments, electrical input from the device and data reception from the sensor have to alternate: the sensor receives data on brain activity and then the sensor shuts off while the electrodes provide stimulation to the brain, and then the electrodes providing stimulation to the brain shut off while the sensor receives data on brain activity.

### Definitions

The term “delta wave” as used herein is interchangeable with and comprises “slow wave” of slow wave sleep (SWS) or slow wave brain activity (SWA). The delta wave is brain activity that occurs at generally higher amperage and corresponds to generally lower frequency brain activity than other measured brain activity and tends to be most pronounced toward the end of Stage 3 sleep and during Stage 4 non-REM deep sleep. The delta wave is often measured topically on the skin with EEG sensors at or above 75 microvolts (μV) and at a frequency between 0.5 Hertz (Hz) and 4 Hz.

The term “subject” or “user” as used herein refers to a living mammal and may be interchangeably used with the term “patient.” Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. The term does not denote a particular age or gender.

As used herein, the term “treating” or “treatment” includes reducing, arresting, preventing, or reversing the symptoms, clinical signs, or underlying pathology of a condition to stabilize or improve a subject's condition or to reduce the likelihood that the subject's condition will worsen as much as if the subject did not receive the treatment. As used herein, “treating” or “treatment” can include improving a subject's condition to increase long-term resilience against factors that can adversely impact the user's health to reduce risks of such exposure, such as factors attributed to adverse environmental conditions (e.g., increased radiation environments associated with deep space travel, trauma, etc.) or activities that can adversely impact the user's health, such as increased physical activity associated with athletic training and competition or medical procedures and treatments with potential for adverse health effects (e.g., surgical operations, radiation treatments, application of medications with potential for adverse side effects, etc.). Treatment may also involve recovery from physiological or psychological damage or disease resulting from exposure to potentially damaging conditions or activities.

As used herein, the term neuroglymphatic electrical stimulation (NGES) refers to support of the glymphatic system in removing toxins from the brain via enhanced pumping and flushing actions of cerebrospinal fluid and interstitial fluid, and accelerated unidirectional flows or pulsatile motions of the fluids, resulting from electrical input to improve constriction and expansion of blood vessels and extracellular spaces. NGES may refer to improving the effectiveness of toxin removal of a functioning glymphatic system or restoring toxin removal functionality to an otherwise mostly non-functioning or entirely non-functioning glymphatic system via the use of electrical input to add kinetic energy to the system.

As used herein, an individual “at risk” of developing a particular disease, disorder, or condition may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of a particular disease, disorder, or condition, as known in the art. An individual having one or more of these risk factors has a higher probability of developing a particular disease, disorder, or condition than an individual without one or more of these risk factors. Risk factors may include exposure to adverse environmental conditions and involvement in activities that correspond with increased potential for physiological and psychological damage or disease.

Exemplary Uses and Methods of the Present Invention

Electroencephalography (EEG) records the neural activity of electrical potential across cell membranes, which are detected through the cortex and recorded by a plurality of scalp electrodes. Changes in electrical potential in the cortex associated with the non-rapid eye movement (NREM) deep sleep delta brainwave (delta wave) contain rhythmical activity, which typically occur at relatively stronger intensity, relatively slower frequencies of 0.5 Hz to 20 Hz, and of generally more consistent and more well-defined wave structure and form than other brainwaves. While awake, fast, less consistent and well-structured signals are predominately generated at lower intensities and at higher frequencies.

Five distinct brain wave patterns are commonly detected during an EEG recording: alpha waves, beta waves, gamma waves, theta waves and delta waves.

In one aspect, a method for modulating delta brainwaves, comprising a) detecting and monitoring an initial delta wave in a subject; b) applying an electrical current to modulate the initial delta wave in the subject, thereby modifying the initial delta wave in the subject to form a modified delta wave; c) optionally repeating cycles of steps a) and b), thereby modulating delta brainwaves, is provided herein. In another aspect, a method for modulating delta brainwaves in a sleeping subject, comprising a) detecting and monitoring an initial delta wave in the sleeping subject; b) applying an electrical current to modulate the initial delta wave in the sleeping subject, thereby modifying the initial delta wave in the sleeping subject to form a modified delta wave; c) optionally repeating cycles of steps a) and b), thereby modulating delta brainwaves in the sleeping subject, is provided herein.

Numerous embodiments are further provided that can be applied to any aspect of the present invention described herein. For example, in some embodiments, the delta brainwaves are monitored by EEG. In some embodiments, the initial delta wave in the subject is detected during NREM sleep. In some embodiments, the initial delta wave in the subject is detected during stage 2 or 3 NREM sleep. In some embodiments, the initial delta wave in the subject is detected during slow-wave sleep. In some embodiments, the initial delta wave in the subject has a frequency of less than about 20 hertz (Hz), less than about 10 Hz, less than about 4 Hz, less than about 2 Hz, or less than about 0.5 Hz. In some embodiments, the initial delta wave in the subject has a frequency selected from the group consisting of about 5 Hz, about 4.5 Hz, about 4 Hz, about 3.9 Hz, about 3.8 Hz, about 3.7 Hz, about 3.6 Hz, about 3.5 Hz, about 3.4 Hz, about 3.3 Hz, about 3.2 Hz, about 3.1 Hz, about 3 Hz, about 2.9 Hz, about 2.8 Hz, about 2.7 Hz, about 2.6 Hz, about 2.5 Hz, about 2.4 Hz, about 2.3 Hz, about 2.2 Hz, about 2.1 Hz, about 2 Hz, about 1.9 Hz, about 1.8 Hz, about 1.7 Hz, about 1.6 Hz, about 1.5 Hz, about 1.4 Hz, about 1.3 Hz, about 1.2 Hz, about 1.1 Hz, about 1 Hz, about 0.9 Hz, about 0.8 Hz, about 0.7 Hz, about 0.6 Hz, about 0.5 Hz, about 0.4 Hz, about 0.3 Hz, about 0.2 Hz, about 0.1 Hz, and about 0 Hz. In some embodiments, the initial delta wave in the subject is not detectable by EEG.

In some embodiments, the electrical current is less than about 30 milliamps (mA), less than about 27 mA, less than about 24 mA, less than about 12 mA, less than about 10 mA, less than about 5 mA, less than about 2 mA, less than about 1 mA, or less than about 0.5 mA. In some embodiments, the electrical current is selected from the group consisting of about 10 mA, about 9.9 mA, about 9.8 mA, about 9.7 mA, about 9.6 mA, about 9.5 mA, about 9.4 mA, about 9.3 mA, about 9.2 mA, about 9.1 mA, about 9 mA, about 8.9 mA, about 8.8 mA, about 8.7 mA, about 8.6 mA, about 8.5 mA, about 8.4 mA, about 8.3 mA, about 8.2 mA, about 8.1 mA, about 8 mA, about 7.9 mA, about 7.8 mA, about 7.7 mA, about 7.6 mA, about 7.5 mA, about 7.4 mA, about 7.3 mA, about 7.2 mA, about 7.1 mA, about 7 mA, about 6.9 mA, about 6.8 mA, about 6.7 mA, about 6.6 mA, about 6.5 mA, about 6.4 mA, about 6.3 mA, about 6.2 mA, about 6.1 mA, about 6 mA, about 5.9 mA, about 5.8 mA, about 5.7 mA, about 5.6 mA, about 5.5 mA, about 5.4 mA, about 5.3 mA, about 5.2 mA, about 5.1 mA, about 5 mA, about 5.9 mA, about 5.8 mA, about 5.7 mA, about 5.6 mA, about 5.5 mA, about 5.4 mA, about 5.3 mA, about 5.2 mA, about 5.1 mA, about 5 mA, about 4.9 mA, about 4.8 mA, about 4.7 mA, about 4.6 mA, about 4.5 mA, about 4.4 mA, about 4.3 mA, about 4.2 mA, about 4.1 mA, about 4 mA, about 3.9 mA, about 3.8 mA, about 3.7 mA, about 3.6 mA, about 3.5 mA, about 3.4 mA, about 3.3 mA, about 3.2 mA, about 3.1 mA, about 3 mA, about 2.9 mA, about 2.8 mA, about 2.7 mA, about 2.6 mA, about 2.5 mA, about 2.4 mA, about 2.3 mA, about 2.2 mA, about 2.1 mA, about 2 mA, about 1.9 mA, about 1.8 mA, about 1.7 mA, about 1.6 mA, about 1.5 mA, about 1.4 mA, about 1.3 mA, about 1.2 mA, about 1.1 mA, about 1 mA, about 0.9 mA, about 0.8 mA, about 0.7 mA, about 0.6 mA, about 0.5 mA, about 0.4 mA, about 0.3 mA, about 0.2 mA, and about 0.1 mA. In some embodiments, the electrical current is about 0.1 mA to about 5 mA, preferably about 2 mA.

In some embodiments, the electrical voltage is less than about 80 to 160 volts (V), less than about 81V, less than about 54V, less than about 27V, less than about 13.5V, less than about 5.4V, less than about 2.7V, less than about 1.4V, less than about 0.7V, and less than about 0.3V.

In some embodiments, the method further comprises measuring a second frequency of the modified delta wave. In some embodiments, the modified delta wave in the subject has a frequency of less than about 20 Hz, less than about 15 Hz, less than about 12 Hz, less than about 7 Hz, or less than about 4 Hz. In some embodiments, the modified delta wave in the subject has a frequency selected from the group consisting of about 20 Hz, about 19.5 Hz, about 19 Hz, about 18.5 Hz, about 18 Hz, about 17.5 Hz, about 17 Hz, about 16.5 Hz, about 16 Hz, about 15.5 Hz, about 15 Hz, about 14.5 Hz, about 14 Hz, about 13.5 Hz, about 13 Hz, about 12.5 Hz, about 12 Hz, about 11.5 Hz, about 11 Hz, about 10.5 Hz, about 10 Hz, about 9.5 Hz, about 9 Hz, about 8.5 Hz, about 8 Hz, about 7.5 Hz, about 7 Hz, about 6.9 Hz, about 6.8 Hz, about 6.7 Hz, about 6.6 Hz, about 6.5 Hz, about 6.4 Hz, about 6.3 Hz, about 6.2 Hz, about 6.1 Hz, about 6 Hz, about 5.9 Hz, about 5.8 Hz, about 5.7 Hz, about 5.6 Hz, about 5.5 Hz, about 5.4 Hz, about 5.3 Hz, about 5.2 Hz, about 5.1 Hz, about 5 Hz, about 4.9 Hz, about 4.8 Hz, about 4.7 Hz, about 4.6 Hz, about 4.5 Hz, about 4.4 Hz, about 4.3 Hz, about 4.2 Hz, about 4.1 Hz, about 4 Hz, about 3.9 Hz, about 3.8 Hz, about 3.7 Hz, about 3.6 Hz, about 3.5 Hz, about 3.4 Hz, about 3.3 Hz, about 3.2 Hz, about 3.1 Hz, about 3 Hz, about 4 Hz, about 3.9 Hz, about 3.8 Hz, about 3.7 Hz, about 3.6 Hz, about 3.5 Hz, about 3.4 Hz, about 3.3 Hz, about 3.2 Hz, about 3.1 Hz, about 3 Hz, about 3.9 Hz, about 3.8 Hz, about 3.7 Hz, about 3.6 Hz, about 3.5 Hz, about 3.4 Hz, about 3.3 Hz, about 3.2 Hz, about 3.1 Hz, about 3 Hz, about 2.9 Hz, about 2.8 Hz, about 2.7 Hz, about 2.6 Hz, about 2.5 Hz, about 2.4 Hz, about 2.3 Hz, about 2.2 Hz, about 2.1 Hz, about 2 Hz, about 1.9 Hz, about 1.8 Hz, about 1.7 Hz, about 1.6 Hz, about 1.5 Hz, about 1.4 Hz, about 1.3 Hz, about 1.2 Hz, about 1.1 Hz, about 1 Hz, about 0.9 Hz, about 0.8 Hz, about 0.7 Hz, about 0.6 Hz, about 0.5 Hz, about 0.4 Hz, about 0.3 Hz, about 0.2 Hz, about 0.1 Hz, and about 0 Hz. In some embodiments, the modified delta wave in the subject has a frequency of about 0.1 Hz to about 5 Hz, about 0.5 Hz to about 4 Hz, or about 0.5 Hz to about 2 Hz. In other embodiments, the modified delta wave in the subject is detectable by EEG.

In some embodiments, the method further comprises monitoring one or more of the following alpha brainwaves, beta brainwaves, gamma brainwaves, and theta brainwaves in the subject. In some embodiments, the modified delta wave in the subject is sinusoidal or its structure becomes more sinusoidal. In some embodiments, the modified delta wave in the subject synchronizes the delta brainwaves in the subject. In some embodiments, the delta brainwaves in the subject are entrained.

In another aspect, a method of treating a disease or a condition, comprising modulating delta brainwaves in a subject in need thereof, comprising a) detecting and monitoring an initial delta wave in the subject; b) applying an electrical current to modulate the initial delta wave in the subject, thereby modifying the initial delta wave in the subject to form a modified delta wave; c) optionally repeating cycles of steps a) and b), thereby treating the disease or the condition, is provided herein. In still another aspect, a method of treating a disease or a condition, comprising modulating delta brainwaves in a sleeping subject in need thereof, comprising a) detecting and monitoring an initial delta wave in the sleeping subject; b) applying an electrical current to modulate the initial delta wave in the sleeping subject, thereby modifying the initial delta wave in the sleeping subject to form a modified delta wave; c) optionally repeating cycles of steps a) and b), thereby treating the disease or the condition, is provided herein.

In another aspect, a method of reducing or preventing a disease or a condition, comprising modulating delta brainwaves in a subject in need thereof, comprising a) detecting and monitoring an initial delta wave in a subject; b) applying an electrical current to modulate the initial delta wave in the subject, thereby modifying the initial delta wave in the subject to form a modified delta wave; c) optionally repeating cycles of steps a) and b), thereby reducing or preventing the disease or the condition, is provided herein. In yet another aspect, a method of reducing or preventing a disease or a condition, comprising modulating delta brainwaves in a sleeping subject in need thereof, comprising a) detecting and monitoring an initial delta wave in the sleeping subject; b) applying an electrical current to modulate the initial delta wave in the sleeping subject, thereby modifying the initial delta wave in the sleeping subject to form a modified delta wave; c) optionally repeating cycles of steps a) and b), thereby reducing or preventing the disease or the condition, is provided herein.

In still another aspect, treating a disease or a condition by restoring the delta wave of a subject in whom an initial delta wave is no longer detectable, comprising modulating delta brainwaves in a sleeping subject in need thereof, comprising a) detecting and monitoring sleep metrics selected from the group consisting of heart rate, breathing rate, and body motion and the initial delta wave; b) applying an electrical current to initiate formation of and to modulate the initial delta wave in the sleeping subject, thereby initiating and modifying the delta wave in the sleeping subject to form a modified delta wave; c) optionally repeating cycles of steps a) and b), thereby treating the disease or the condition, is provided herein.

As described above, embodiments are applicable to any method described herein. For example, in some embodiments, the disease or the condition is selected from the group consisting of alcoholism, Alzheimer's disease, anxiety, attention deficit hyperactivity disorder, autism, behavioral disorders, bone damage, cancer, cataracts, chronic or acute pain, chronic traumatic encephalopathy (CTE), depression, diabetes, digestive and metabolism-related disorders, fibromyalgia, hormonal imbalances, immune system disorders, inflammation, an ischemic brain disease, juvenile chronic arthritis, narcolepsy, obsessive-compulsive disorder, a parasomnia, Parkinson's disease, radiation exposure, schizophrenia, post-traumatic stress disorder (PTSD), sleep deficiencies, stroke, temporal lobe epilepsy, and tissue damage. In some embodiments, the disease or condition is a poorly functioning or non-functioning glymphatic system. In some embodiments, the disease or the condition is radiation exposure. In some embodiments, the radiation exposure is acute, chronic, in outer space, solar, or interstellar. In some embodiments, the radiation exposure is in Earth's atmosphere at altitudes above one mile. In some embodiments, the radiation exposure is in outer space, including low Earth orbit and deep space. In some embodiments, the disease or condition is a physiological disorder, damage, or infection by pathogen. In some embodiments, no settings may be changed across diseases and conditions. In some embodiments, the systems and devices described herein treat and/or prevent each of the diseases in the same manner: by maximizing the health of the delta wave. In some embodiments, the device may provide input to entirely recreate the delta wave when no delta wave is detected (e.g., late stage Alzheimer's).

In some embodiments, the disease or the condition is a neurological disorder. In some embodiments, the neurological disorder is selected from the group consisting of Alzheimer's disease, anxiety, attention deficit hyperactivity disorder, autism, chronic or acute pain, chronic traumatic encephalopathy (CTE), depression, an ischemic brain disease, narcolepsy, Parkinson's disease, post-traumatic stress disorder (PTSD), sleep deficiencies, stroke, and temporal lobe epilepsy. In some embodiments, the neurological disorder is selected from the group consisting of Alzheimer's disease, attention deficit hyperactivity disorder, narcolepsy, and temporal lobe epilepsy. In some embodiments, the conditions are associated with exposure to harmful environments or engagement in activities that can damage tissues or bones or increase potential for disease, such as athletic training or competition, or medical procedures, treatments, or medications and the duration of the treatment by this method may be temporary and defined according to the time interval of the exposure to the adverse environment or activity.

In some embodiments, the method is administered daily, e.g., during sleep. In some embodiments, the method is administered for only one sleep period. In some embodiments, the method is administered during sleep periods over the course of two or more days. In some embodiments, the method is administered at least daily for at least 2 weeks, at least daily for at least 4 weeks, at least daily for at least 6 weeks, at least daily for at least 8 weeks, at least daily for at least 2 months, at least daily for at least 4 months, or at least daily for at least 6 months. In some embodiments, the method is administered daily during exposure to an adverse environmental factor or activity with potential to increase likelihood of physiological or psychological damage or disease. In some embodiments, the method is administered daily to reduce or to prevent a disease or a condition. In some embodiments, the method is administered at least one day prior to, during, and at least one day after exposure to an adverse environmental factor or activity with potential to increase likelihood of physiological or psychological damage or disease. In some embodiments, the method is administered at least one day prior to, during, and at least one day after the disease or the condition are detected. In some embodiments, duration of use is situation dependent. In some embodiments, users may use the device to treat a disease or condition and stop use once the disease or condition no longer exists. In some embodiments, users may use the device only throughout the duration of exposure to a damaging environment to help prevent disease or condition (e.g., increased radiation during deep space travel or during periods of time of extreme physical exertion and increased likelihood of personal injury, such as football players during a football season).

Exemplary Devices of the Present Invention

In some embodiments, a mobile battery powered device to overcome solar and interstellar radiation impacts with sleep-brainwave entrainment (OSIRIS-BE) restores the non-rapid eye movement (NREM) deep sleep delta brainwave's (delta wave's) intensity, sinusoidal structure, and duration to enhance the physiologically and cognitively restorative benefits initiated by the delta wave to increase human resilience to the effects of radiation. In some embodiments, OSIRIS-BE electrically stimulates the nervous and glymphatic systems with a small unit applying electrical current through electrodes topically applied to the front (just above bridge of nose at the middle of the frontal lobes; negative electrode) and back of the head (positive electrode) or to the front of the head (above bridge of nose at the middle of the frontal lobe [negative electrode], and at upper bridge of nose [positive electrode]). In some embodiments, OSIRIS-BE monitors brainwave activity with an electroencephalogram (EEG) to monitor the delta wave onset and structure and monitors other sleep state indicators identifying onset of NREM deep sleep (e.g., heart rate, breathing rate, body motion). In some embodiments, OSIRIS-BE amplifies and improves the user's delta wave structure at the frequency of the delta wave of the user as it occurs in the user during the sleep period of use. In some embodiments, OSIRIS-BE uses an algorithm to apply electrical current at the appropriate times and intensities (in analog) of NREM deep sleep, based on the EEG and sleep state monitoring data, to provide targeted enhancement and restoration of the delta wave to optimal intensity, sinusoidal wave structure, and duration such as that of the user's healthy early childhood delta wave. In some embodiments, OSIRIS-BE monitors brainwaves for the emergence of low frequency (˜0.1 Hertz [Hz] to 4 Hz, and as high as 20 Hz), high amplitude (generally above 75 μV, but also sometimes as low as less than 10 μV and greater than 140 μV, as detected topically on the skin) brainwaves during sleep. In some embodiments, OSIRIS-BE applies an algorithm to identify the delta wave based on the monitoring data, assess the delta wave's health based on the data (frequency in Hz, intensity in μV as measured topically on skin, and wave structure integrity), identify its healthy or ideal natural best-state based on the assessment, and input electrical current to gradually entrain the delta wave back to its healthy or ideal natural best-state during the user's non-REM deep sleep cycles. In some embodiments, the duration of the monitored delta wave activity and the duration of electrical input is timed by OSIRIS-BE during each sleep session such that the total combined duration of electrical input for each sleep session never exceeds 2 hours and the electrical input for each non-REM deep sleep cycle never exceeds a total duration of 40 minutes. In some embodiments, in order to not interfere with the progression of sleep cycles through natural sleep stages (non-REM through REM and back to non-REM) the intensity of electrical input also diminishes and eventually ceases as the measured delta wave diminishes and becomes undetectable toward the end of the sleep stage. In some embodiments, the electrical input once again begins to be applied as the delta wave becomes detectable when the user cycles into the next non-REM deep sleep stage. In order to avoid interference between inputs to OSIRIS-BE (monitoring data) and outputs from the OSIRIS-BE (electrical current), OSIRIS-BE does not output electrical current at the same time it is receiving input and instead alternates between providing output and receiving input. In some embodiments, the algorithm adjusts the output from the device between each output/input cycle based on effectiveness of the device's output in achieving intended results based on input. In some embodiments, the output electrical current from the device matches the frequency of the user's delta wave and is designed to rise and fall (in analog/sine wave, not digital square wave) in amplitude at this frequency slightly above the amplitude of the user's measured delta wave, adjusting for current loss between brain and skin surface, in order to gradually entrain the delta wave to healthy or ideal natural best-state in sync with progression through sleep cycles during each sleep session (full sleep period). In some embodiments, OSIRIS-BE provides electrical current to reconstruct the delta wave at the appropriate times of NREM deep sleep for users who no longer have a detectable delta wave due to brain damage, trauma, disease, or other factors, such as neurodegeneration as in individuals in advanced stages of Alzheimer's disease. In some embodiments, the electrical current from the device is applied by the device once the sleep metrics data have adequately identified the non-REM deep sleep stages (Stages 3 and 4) as separate from the REM sleep stages, and the device has not been able to detect a delta wave sufficiently to identify its frequency in the user. In some embodiments, the electrical current output from the device to the user is provided at the end of Stage 3 and at Stage 4 sleep and cycles through frequencies (from low to high) and intensities (from low to high), with each output cycle beginning at ˜0.1 Hz from low to rising voltage, and the next output cycle beginning at ˜0.2 Hz from low to rising voltage, and so on up to ˜4 Hz. In some embodiments, the device monitors brainwave activity from the user after each output cycle from the device to assess responsiveness of the user's brainwave activity to the device's output in order to identify the best-fit frequency. In some embodiments, best-fit frequency is identified by measured duration and intensity of delta wave activity from the user following each device output cycle, with the longest duration and highest intensity brainwave feedback from the user corresponding to the user's proper delta wave frequency. In some embodiments, in the absence of a discernable best-fit frequency based on these cycles, the device initially provides output at a standard frequency of 0.5 Hz and continues to monitor for delta wave activity from the user, adjusting device output accordingly.

A device, comprising a) at least one sensor to monitor brainwave activity including a delta wave; b) an amplifier and control board; c) a negative electrode; d) a positive electrode; and e) an electrical stimulator to modulate delta brainwaves, is provided herein. In another aspect, a device, comprising a) at least one sensor to monitor sleep brainwave activity including a delta wave; b) an amplifier and control board; c) a negative electrode; d) a positive electrode e) an electrical stimulator to modulate sleep brainwaves, is provided herein.

In yet another aspect, a device, comprising a) at least one sensor to monitor brainwave activity including a delta wave; b) at least one sensor to monitor health metrics selected from the group consisting of heart rate, breathing rate, and body motion; c) an amplifier and control board; d) a negative electrode; e) a positive electrode; and f) an electrical stimulator to initiate and modulate delta brainwaves, is provided herein.

As described above, embodiments are applicable to any device described herein. For example, in some embodiments, the device further comprises a sensor component, wherein the sensor component comprises the sensor to monitor brainwave activity including the delta wave. In some embodiments, the sensor component is configured to contact a head of a subject. In some embodiments, the sensor component is in a headband. In some embodiments, the sensor component, electrodes, and other device components are imbedded in a flexible material that attaches to the front of the head like a sticker via the adhesive properties of electrically conductive hydrogel. In some embodiments, the sensor component comprises an EEG sensor. In some embodiments, the sensor component further comprises the negative electrode and the positive electrode. In some embodiments, the device further comprises a housing unit. In some embodiments, the housing unit is connected by a wire to the sensor component. In some embodiments, the housing unit is connected by one or more wires to the negative electrode and the positive electrode. In some embodiments, the housing unit is plastic. In some embodiments, the electrical stimulator delivers an electrical current. In some embodiments, the delivered electrical current is less than about 30 mA (e.g., 10 mA) or is about 2 mA. In some embodiments, the device modulates an initial delta wave in the subject, thereby modifying the initial delta wave in a subject to form a modified delta wave. In some embodiments, the modified delta wave in the subject has a frequency of less than about 20 Hz, less than about 15 Hz, less than about 4 Hz, or about 0.5 Hz to about 2 Hz.

In one aspect, a device for use in modulating delta brainwaves is provided herein. In another aspect, a device for use in treating a disease or a condition, comprising modulating delta brainwaves is provided herein. In another aspect, a device for use in reducing or preventing a disease or a condition, comprising modulating delta brainwaves is provided herein. In another aspect, a device for use in accelerating recovery from physiological, neurological, or psychological damage or disease, comprising modulating delta brainwaves is provided herein. In some embodiments, the use comprises a) detecting and monitoring an initial delta wave in a subject; b) applying an electrical current to modulate the initial delta wave in the subject, thereby modifying the initial delta wave in the subject to form a modified delta wave; c) optionally repeating cycles of steps a) and b), thereby modulating delta brainwaves.

In another aspect, a device for use in modulating delta brainwaves in a sleeping subject is provided herein. In another aspect, a device for use in treating a disease or a condition, comprising modulating delta brainwaves in a sleeping subject is provided herein. In another aspect, a device for use in reducing or preventing a disease or a condition, comprising modulating delta brainwaves in a sleeping subject. In another aspect, a device for use in accelerating recovery from physiological or psychological damage or disease, comprising modulating delta brainwaves in a sleeping subject. In some embodiments, the use comprises a) detecting and monitoring an initial delta wave in the sleeping subject; b) applying an electrical current to modulate the initial delta wave in the sleeping subject, thereby modifying the initial delta wave in the sleeping subject to form a modified delta wave; c) optionally repeating cycles of steps a) and b), thereby modulating delta brainwaves in the sleeping subject.

In yet another aspect, a device for use in initiating and modulating delta brainwaves in a sleeping subject is provided herein. In another aspect, a device for use in treating a disease or a condition, comprising initiating and modulating delta brainwaves in a sleeping subject is provided herein. In another aspect, a device for use in reducing or preventing a disease or a condition, comprising initiating and modulating delta brainwaves in a sleeping subject. In another aspect, a device for use in accelerating recovery from physiological or psychological damage or disease, comprising initiating and modulating delta brainwaves in a sleeping subject. In some embodiments, the use comprises a) detecting and monitoring sleep metrics selected from the group consisting of heart rate, breathing rate, and body motion and an initial delta wave in the sleeping subject; b) applying an electrical current to initiate formation of and to modulate the initial delta wave in the sleeping subject, thereby initiating or modifying the initial delta wave in the sleeping subject to form a modified delta wave; c) optionally repeating cycles of steps a) and b), thereby modulating delta brainwaves in the sleeping subject.

### EXAMPLES

### Example 1: Overcome Solar and Interstellar Radiation Impacts with Sleep-Brainwave Entrainment (OSIRIS-BE)

A mobile battery powered device to overcome solar and interstellar radiation impacts with sleep brainwave entrainment (OSIRIS-BE) restores the non-rapid eye movement (NREM) deep sleep delta brainwave's (delta wave's) intensity, sinusoidal structure, and duration to enhance the physiologically and cognitively restorative benefits initiated by the delta wave to increase human resilience to the effects of radiation. OSIRIS-BE electrically stimulates the nervous and/or glymphatic systems with a small unit applying electrical current through electrodes topically applied to the front (just above bridge of nose at the middle of the frontal lobes; negative electrode) and back of the head (positive electrode) or to the front of the head (above bridge of nose at the middle of the frontal lobe [negative electrode], and at upper bridge of nose [positive electrode]). OSIRIS-BE monitors brainwave activity with an electroencephalogram (EEG) type of system to monitor the delta wave onset and structure and monitors other sleep state indicators identifying onset of NREM deep sleep (heart rate, breathing rate, body motion). OSIRIS-BE uses an algorithm to apply electrical current at the appropriate times and intensities (in analog) of NREM deep sleep, based on the EEG and sleep state monitoring data, to provide targeted enhancement and restoration of the delta wave to optimal intensity, sinusoidal wave structure, and duration such as that of the user's healthy early childhood delta wave. OSIRIS-BE provides electrical current to initiate by stimulation and reconstruct the delta wave with the activation and recruitment of neurons at the appropriate durations and times of NREM sleep for users who no longer have a detectable delta wave due to brain damage, trauma, disease, or other factors or conditions.

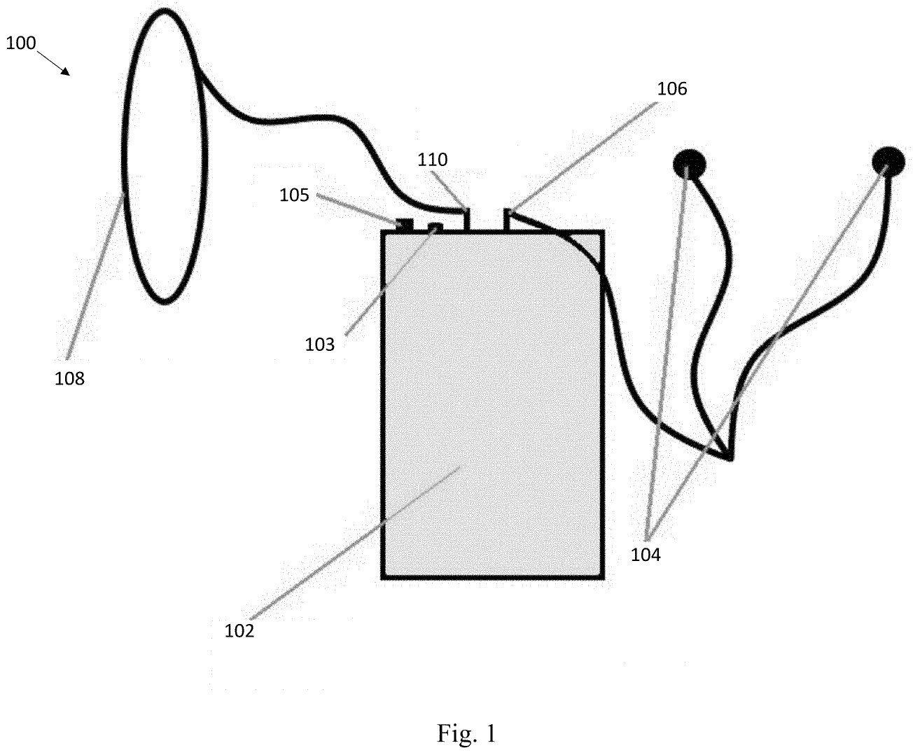

FIG. 1 shows a device 100 illustration of Overcoming Solar and Interstellar Radiation Impacts with Sleep Brainwave Entrainment (OSIRIS-BE) Device. OSIRIS-BE Device pairs with mobile device (phone, tablet, etc.) wirelessly (e.g., via Bluetooth) and interfaces with mobile device via OSIRIS-BE Device Application installed on the mobile device. OSIRIS-BE sends data to the paired mobile device. Data sent to the paired mobile device consists of system check information, battery level information, information from the brainwave sensors (when electrodes used or not used), information from the electrodes when used, and other system and use information. The data is presented in the OSIRIS-BE Device Application installed on the mobile device. OSIRIS-BE Device automatically resets itself and operates entirely independently of the OSIRIS-BE Device Application and mobile device using the on/off switch and with the on/off light providing critical device data needed for “no data” (basic) use mode.

As shown in FIG. 1, the device 100 includes a main housing 102. In various embodiments, the housing 102 may contain one or more electrical components (e.g., processor, battery components, controller, circuit board components, and/or wiring). In various embodiments, the housing may include an on/off light 103. In various embodiments, the housing may include an on/off switch 105. The device further includes two or more electrodes 104 extending therefrom. In various embodiments, the electrodes 104 include at least one positive electrode. In various embodiments, the electrodes 104 include at least one negative electrode. In various embodiments, the positive electrode may be placed on the back of the head and/or base of the head just below the hair line. In various embodiments, the negative electrode may be placed on the front of the head (e.g., the forehead). In various embodiments, each of the electrodes 104 may include a pad configured to be attached to skin. In various embodiments, the pad may include an adhesive. The electrodes 104 may be coupled to the housing 102 via an electrode jack 106.

In various embodiments, the device 100 may further include one or more brainwave sensors 108 coupled to the housing 102 via a wire jack 110. In various embodiments, the brainwave sensor(s) 108 may be contained within a headband configured to fit around a head. In various embodiments, the headband may contain the two or more electrodes 104. In the example where the two or more electrodes 104 are disposed on or within the headband, the two or more electrodes 104 may not require any adhesive as described above as the headband may be used to secure the electrodes 104 against the skin.

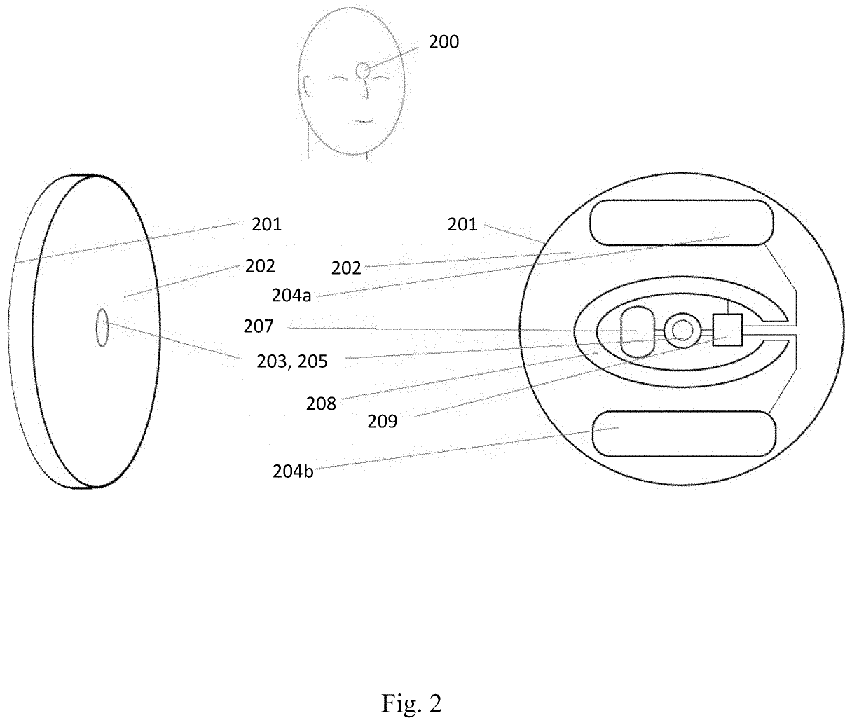

FIG. 2 shows device illustration of OSIRIS-BE Device 200 in a compact version that does not involve external wires. The OSIRIS-BE Device may include similar components to the OSIRIS-BE Device shown in FIG. 1. In particular, the OSIRIS-BE Device may include a flexible (e.g., rubber) housing having a first side configured to be coupled with (e.g., adhered to) the skin of a user and a second side opposite the first side. The housing may house various electrical components including, for example, one or more electrodes, a battery, an on/off button, a brainwave sensor, a control board, an amplifier, and/or a data processor. In various embodiments, the housing may further include a wireless chip (e.g., Bluetooth) configured to provide wireless communications with another device (e.g., a mobile device).

In various embodiments, the device 200 may be worn during sleep topically on the skin. In various embodiments, the device 200 may include an adhesive. In various embodiments, the device 200 may include an electrically conductive fluid 201 (e.g., conductive hydrogel) located on the underside of the device 200 such that the conductive fluid makes contact with the skin. In various embodiments, the device 200 may include an on/off light 203. In various embodiments, the housing may include an on/off switch 205. In various embodiments, the device 200 may be placed such that the bottom of the device is at the upper bridge of the nose and the top of the device is above the bridge of the nose. In various embodiments, the device 200 may be placed centrally on the forehead (between the frontal lobes). In various embodiments, the top of the device may correspond to the negative electrode 204a. In various embodiments, the bottom of the device may correspond to the positive electrode 204b. In various embodiments, the device 200 may include a flexible casing 202 (e.g., polymer, rubber, etc.). In various embodiments, the device 200 may include a battery 207. In various embodiments, the device 200 may include an electrical components 209. In various embodiments, the electrical components 209 include at least one of: a control board, an amplifier, a data processor, and/or a Bluetooth chip. In various embodiments, the device may have a diameter (or length) of about 0.5 inches to about 2 inches, preferably about 1 inch.

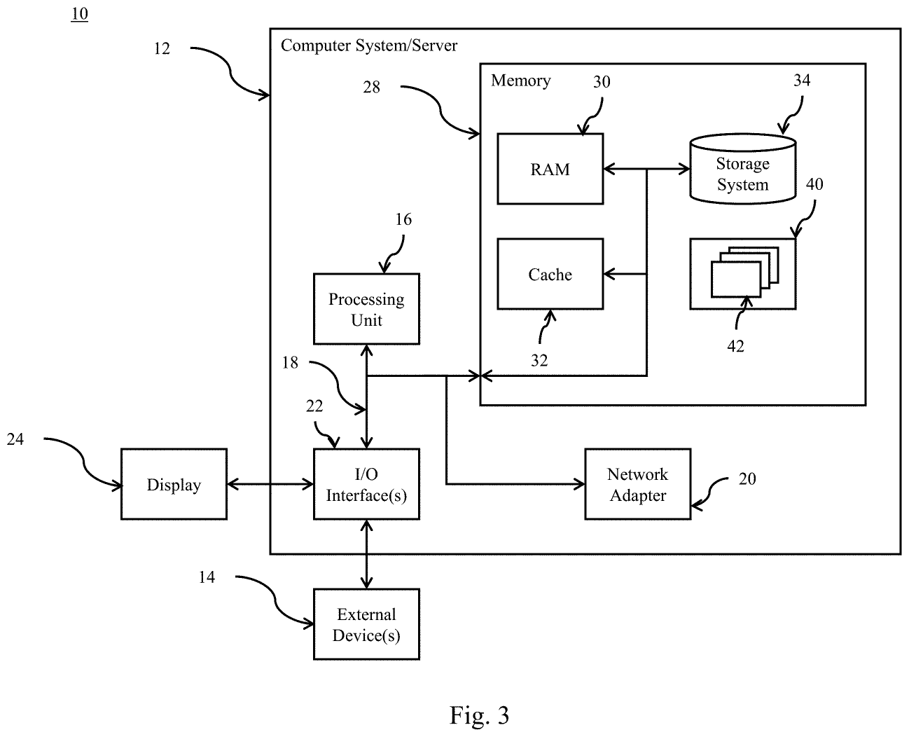

Referring now to FIG. 3, a schematic of an example of a computing node is shown. Computing node 10 is only one example of a suitable computing node and is not intended to suggest any limitation as to the scope of use or functionality of embodiments described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 3, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the disclosure.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method for modulating delta waves of a brain, comprising:
a) detecting and monitoring an initial delta wave in a subject;
b) applying an electrical current to modulate the initial delta wave in the subject, thereby modifying the initial delta wave in the subject to form a modified delta wave;
c) optionally repeating cycles of steps a) and b), thereby modulating delta waves.

2. A method for modulating delta waves of a brain in a sleeping subject, comprising:
a) detecting and monitoring an initial delta wave in the sleeping subject;
b) applying an electrical current to modulate the initial delta wave in the sleeping subject, thereby modifying the initial delta wave in the sleeping subject to form a modified delta wave;
c) optionally repeating cycles of steps a) and b), thereby modulating delta waves in the sleeping subject.

3. (canceled)

4. The method of claim 1, wherein the delta waves are monitored by electroencephalography (EEG).

5. (canceled)

6. The method of claim 1, wherein the initial delta wave in the subject is detected during stages 2 or 3 non-rapid eye movement (NREM) sleep.

7-11. (canceled)

12. The method of claim 1, wherein the initial delta wave in the subject is not detectable by EEG.

13-17. (canceled)

18. The method of claim 1, wherein the electrical current is about 0.1 mA to about 5 mA.

19. (canceled)

20. The method of claim 1, wherein the electrical voltage is 80 to 160 volts (V), less than about 81V, less than about 54V, less than about 27V, less than about 13.5V, less than about 5.4V, less than about 2.7V, less than about 1.4V, less than about 0.7V, or less than about 0.3V.

21-26. (canceled)

27. The method of claim 1, wherein the modified delta wave in the subject has a frequency from about 0.5 Hz to about 4 Hz.

28. (canceled)

29. The method of claim 1, wherein the modified delta wave in the subject is detectable by EEG.

30. The method of claim 1, further comprising monitoring one or more of the following: alpha waves, beta waves, gamma waves, and theta waves in the subject.

31. The method of claim 1, wherein the modified delta wave in the subject is sinusoidal or its structure becomes more sinusoidal.

32. The method of claim 1, wherein the modified delta wave in the subject synchronizes the delta waves in the subject.

33. The method of claim 1, wherein the delta waves in the subject are entrained.

34-40. (canceled)

41. The method of claim 1, wherein the disease or the condition is selected from the group consisting of: alcoholism, Alzheimer's disease, anxiety, attention deficit hyperactivity disorder (ADHD), autism, behavioral disorders, bone damage, cancer, cataracts, chronic or acute pain, chronic traumatic encephalopathy (CTE), depression, diabetes, digestive and metabolism-related disorders, fibromyalgia, hormonal imbalances, immune system disorders, inflammation, an ischemic brain disease, juvenile chronic arthritis, narcolepsy, obsessive-compulsive disorder, a parasomnia, Parkinson's disease, post-traumatic stress disorder (PTSD), radiation exposure, schizophrenia, sleep deficiencies, stroke, temporal lobe epilepsy, and tissue damage.

42-50. (canceled)

51. The method of claim 1, wherein the method is administered to a patient daily.

52. The method of claim 1, wherein the method is administered to a patient at least daily for at least 2 weeks.

53. The method of claim 1, wherein the method is administered to a patient at least daily for at least 4 weeks.

54. (canceled)

55. The method of claim 1, wherein the method is administered to a patient at least daily for at least 8 weeks.

56. (canceled)

57. (canceled)

58. (canceled)

59. The method of claim 1, wherein the method is administered to a patient at least daily indefinitely.

60. A device, comprising:
a) at least one sensor configured to monitor delta wave activity in a brain;
b) an amplifier and control board;
c) a negative electrode;
d) a positive electrode; and
e) an electrical stimulator to modulate delta waves.

61-87. (canceled)
